(19) United States
(12) Patent Application Publication
(10) Pub. No.: US 2021/0147801 A1
(43) Pub. Date: May 20, 2021

(54) METHODS FOR INCREASING EXPANSION AND IMMUNOSUPPRESSIVE CAPACITY OF A POPULATION OF CD8+CD45RCLOW/- TREGS

(71) Applicants: INSERM (INSTITUT NATIONAL DE LA SANTÉ ET DE LA RECHERCHE MÉDICALE), Paris (FR); UNIVERSITÉ DE NANTES, Nantes (FR); CENTRE HOSPITALER UNIVERSITAIRE DE NANTES, Nantes (FR)

(72) Inventors: Carole GUILLONNEAU, Nantes (FR); Ignacio ANEGON, Nantes (FR); Séverine BEZIE, Nantes (FR)

(73) Assignees: INSERM (INSTITUT NATIONAL DE LA SANTÉ ET DE LA RECHERCHE MÉDICALE), Paris (FR); UNIVERSITÉ DE NANTES, Nantes (FR); CENTRE HOSPITALER UNIVERSITAIRE DE NANTES, Nantes (FR)

(21) Appl. No.: 16/630,098

(22) PCT Filed: Jul. 12, 2018
(86) PCT No.: PCT/EP2018/068882

§ 371 Date: Jan. 10, 2020

(30) Foreign Application Priority Data
Jul. 13, 2017 (EP) 17305939.5

(51) Int. Cl. C12N 5/0783; A61K 35/17; A61P 37/06

(52) U.S. Cl. CPC C12N 5/0637; A61K 35/17; A61P 37/06; C12N 2501/2315; C12N 2510/00; C12N 2501/515; C12N 2501/2302; C12N 2501/04

(57) ABSTRACT

A population of highly suppressive human CD8⁺CD45RC^{low/-} Tregs is characterized by expressing Foxp3 and producing IFNγ, IL-10, IL-34 and TGFβ to mediate their suppressive activity. Accordingly, methods capable of increasing expansion and immunosuppressive capacity of such a population of CD8+CD45RC^{low/-} Tregs are highly desirable for therapeutic purposes. Rapamycin has been shown to increase the expansion and immunosuppressive capacities of the population of CD8⁺CD45RC^{low/-} Tregs. Accordingly, the method of increasing expansion and immunosuppressive capacity of a population of CD8⁺CD45RC^{low/-} Tregs includes culturing the population of CD8⁺CD45RC^{low/-} Tregs in presence of a rapamycin compound.

### FIELD OF THE INVENTION

The present invention relates to methods for increasing expansion and immunosuppressive capacity of a population of CD8+CD45RC^{low/-} Tregs.

### BACKGROUND OF THE INVENTION

Immunosuppressive regimens have significantly improved long-term graft survival in the last decades but they still cannot prevent the allograft from chronic graft dysfunction and they remain a significant obstacle for the welfare of transplanted patients. Thus, in the last years, improvement of allograft survival has stagnated, mainly because of chronic graft rejection, secondary effects and non-specific immunosuppression¹. The identification in human of regulatory cell populations actively controlling immune responses in transplantation with high suppressive capacity and specificity toward donor antigens has generated revolutionizing therapeutic strategies in a number of diseases with a Treg/effector T cells (Teff) deregulation. The establishment of cellular therapy with regulatory cells has recently emerged has a promising future therapy in autoimmunity as well as bone marrow and solid organ transplantation²⁻⁴. Several studies have demonstrated the importance of antigen recognition by the TCR for survival, stimulation of suppressive function and the superiority of such antigen-experienced Tregs⁵⁻⁸ vs. anti-CD3-anti-CD28 polyclonally stimulated Tregs. Phase I studies in GVHD and solid organ transplantation have started with regulatory cells from different types (different CD4⁺ Tregs, macrophages and DCs) without apparent toxicity, but to date, there are no clinical trials using CD8⁺ Tregs despite abundant literature in animals models⁹⁻¹¹. One limitation for translation of CD8⁺ Tregs in humans might be that Foxp3, a critical gene in the function of CD4⁺ Tregs to efficiently restrain immune responses^{12,13}, is not clearly defined for CD8⁺ Tregs and its expression according to other surface markers or cytokines and function has not been clearly demonstrated for CD8⁺ Tregs in humans^{10,14-16.} Recently a new population of highly suppressive human CD8⁺CD45RC^{low}Tregs was identified (WO2017/042170). This population is characterized as expressing Foxp3 and producing IFNγ, IL-10, IL-34 and TGFβ to mediate their suppressive activity. Accordingly methods capable of increasing expansion and immunosuppressive capacity of such a population of CD8+CD45RC^{low/-} Tregs are highly desirable for therapeutic purposes.

### SUMMARY OF THE INVENTION

The present invention relates to methods for increasing expansion and immunosuppressive capacity of a population of CD8⁺CD45RC^{low/-} Tregs. In particular, the present invention is defined by the claims.

### DETAILED DESCRIPTION OF THE INVENTION

The inventors showed that CD8⁺CD45RC^{low/-} Tregs can be more efficiently expanded in presence of rapamycin. Furthermore rapamycin increase the immunosuppressive capacity of the population.

Accordingly, the first object of the present invention relates to a method of increasing expansion and immunosuppressive capacity of a population of CD8⁺CD45RC^{low/-} Tregs comprising culturing the population of CD8⁺CD45RC^{low/-} Tregs in presence of a rapamycin compound.

As used herein, the term “regulatory T cells” or “Tregs” refers to a subpopulation of T cells which modulate the immune system, maintain tolerance to self-antigens, and abrogate autoimmune diseases. These cells generally suppress or downregulate induction and proliferation of effector T cells.

As used herein, the term “population” refers to a population of cells, wherein the majority (e.g., at least about 50%, preferably at least about 60%, more preferably at least about 70%, and even more preferably at least about 80%) of the total number of cells have the specified characteristics of the cells of interest and express the markers of interest (e.g. a population of human CD8⁺CD45RC^{low/-} Treg cells comprises at least about 50%, preferably at least about 60%, more preferably at least about 70%, and even more preferably at least about 80% of cells which have the highly suppressive functions and which express the particular markers of interest).

As used herein, the term “CD8” (cluster of differentiation 8) well known in the art refers to a transmembrane glycoprotein that serves as a co-receptor for the T cell receptor (TCR). To function, CD8 forms a dimer, consisting of a pair of CD8 chains. The most common form of CD8 in T cells is composed of a CD8-α and CD8-β chain and CD8⁺CD45RC^{low/-} Treg cells express both chains. The naturally occurring human CD8-α protein has an amino acid sequence provided in the UniProt database under accession number P01732. The naturally occurring human CD8-β protein has an amino acid sequence provided in the UniProt database under accession number P10966.

As used herein, the term “CD45” (also known as LCA or PTPRC) refers to a transmembrane glycoprotein existing in different isoforms previously described in Streuli et al., 1996. These distinct isoforms of CD45 differ in their extracellular domain structures which arise from alternative splicing of 3 variable exons coding for part of the CD45 extracellular region. The various isoforms of CD45 have different extracellular domains, but have the same transmembrane and cytoplasmic segments having two homologous, highly conserved phosphatase domains of approximately 300 amino acid residues. The naturally occurring human CD45 protein has an amino acid sequence provided in the UniProt database under accession number P08575. As used herein, the term “CD45RC” refers to the exon 6 splice variant (exon C) of the tyrosine phosphatase CD45. The CD45RC isoform is expressed on B cells, and on subsets of CD4⁺ and CD8⁺ T cells. As used herein, the term “low/−” is general term of the skilled person in cytometry for qualifying the expression level of a surface marker and indicates that the surface marker is expressed at an intermediate level or is null.

In some embodiments, the population of CD8⁺CD45RC^{low/-} Treg cells is genetically modified to encode desired expression products, as will be further described below.

The term “genetically modified” indicates that the cells comprise a nucleic acid molecule not naturally present in non-modified population of CD8⁺CD45RC^{low/-} Treg cells, or a nucleic acid molecule present in a non-natural state in said population of CD8⁺CD45RC^{low/-} Treg cells (e.g., amplified). The nucleic acid molecule may have been introduced into said cells or into an ancestor thereof.

A number of approaches can be used to genetically modify a population of cells, such as virus-mediated gene delivery, non-virus-mediated gene delivery, naked DNA, physical treatments, etc. To this end, the nucleic acid is usually incorporated into a vector, such as a recombinant virus, a plasmid, phage, episome, artificial chromosome, etc. Examples of means by which the nucleic acid carrying the gene may be introduced into the cells include, but are not limited to, microinjection, electroporation, transduction, or transfection using DEAE-dextran, lipofection, calcium phosphate or other procedures known to one skilled in the art.

In some embodiments, the population of CD8⁺CD45RC^{low/-} Treg cells is genetically modified using a vector particle such as a viral vector (or a recombinant virus) or a virus-like particle (VLP). In this embodiment, the heterologous nucleic acid is, for example, introduced into a recombinant virus which is then used to infect population of CD8⁺CD45RC^{low/-} Treg cells. Different types of recombinant viruses can be used, in particular recombinant retroviruses. Retroviruses are preferred vectors since retroviral infection results in stable integration into the genome of the cells. This is an important property because lymphocyte expansion, either in vitro or in vivo after injection into the subject, requires that the transgene is maintained stable during segregation in order to be transmitted at each cell division. Examples of retrovirus types which can be used are retroviruses from the oncovirus, lentivirus or spumavirus family Particular examples of the oncovirus family are slow oncovirus, non oncogene carriers, such as MoMLV, ALV, BLV or MMTV, and fast oncoviruses, such as RSV. Examples from the lentivirus family are HIV, SIV, FIV or CAEV. Techniques for constructing defective recombinant retroviruses have been widely described in the literature (WO 89/07150, WO 90/02806, and WO 94/19478). These techniques usually comprise the introduction of a retroviral vector comprising the transgene into an appropriate packaging cell line, followed by the recovery of the viruses produced, said viruses comprising the transgene in their genome. The Population of CD8+CD45RC^{low/-} Treg cells can be infected with recombinant viruses using various protocols, such as by incubation with a virus supernatant, with purified viruses, by co-culturing said Treg cells with the virus' packaging cells, by Transwell techniques, etc. As used herein, the term “Virus-Like Particle” (VLP) refers to a structure resembling a virus particle. A virus-like particle in accordance with the invention is non-replicative since it lacks all or part of the viral genome, typically and preferably lacks all or part of the replicative and infectious components of the viral genome. The term “non-replicative”, as used herein, refers to being incapable of replicating the genome comprised or not in the VLP. VLP may be prepared according to techniques known in the art and for example as described in the international patent application published under n° WO 02/34893.

The nucleic acid used to genetically modify the population of CD8⁺CD45RC^{low/-} Treg cells may encode various biologically active products, including polypeptides (e.g., proteins, peptides, etc.), RNAs, etc. In a particular embodiment, the nucleic acid encodes a polypeptide having an immuno-suppressive activity. In another embodiment, the nucleic acid encodes a polypeptide which is toxic or conditionally toxic to the cells. Preferred examples include a thymidine kinase (which confers toxicity in the presence of nucleoside analogs), such as HSV-1 TK, a cytosine desaminase, etc.

Another preferred category of nucleic acids are those encoding a T cell receptor or a subunit or functional equivalent thereof such as a chimeric antigen receptor (CAR) specific to an antigen of interest or a chimeric autoantibody receptor (CAAR) comprising an auto-antigen. For instance, the expression of recombinant TCRs or CARs specific for an antigen produces human CD8⁺CD45RC^{low/-} Treg cells which can act more specifically and efficiently on effector T cells to inhibit immune responses in a patient in need thereof. The basic principles of chimeric antigen receptor (CAR) design have been extensively described (e.g. Sadelain et al., 2013). CARs comprise an extracellular antigen-recognition moiety generally linked via spacer/hinge and a transmembrane domain to an intracellular signaling domain. The intracellular signaling domain of “first generation” CARs only comprise a T-cell activation moiety. The intracellular domain of “second generation” CARs comprise a co-stimulatory moiety in tandem with an activation moiety, for example CD3. Examples of co-stimulatory domains include, but are not limited to, ICOS, OX40 (CD134), CD28, 4-1BB (CD137), CD27 and DAP10. The intracellular domain of “third generation” CARs comprise two co-stimulatory domains in tandem with an activation moiety, such as the combination of CD28, a tumor necrosis factor receptor (TNFr), such as OX40 or 4-1BB, and CD3 CARs are generally obtained by fusing the extracellular antigen-binding domain with the intracellular signaling domains derived from the CD3-ζ chain of the T-cell receptor, in tandem with costimulatory endo-domains to support survival and proliferative signals. Because CAR-modified T cells function independently of a patient's MHC and can readily be generated for clinical use, the targeting of pathogenic antigens as described below with a CAR based-approach is useful. CAARs comprise an extracellular autoantigen, such as an autoantigen involved in an autoimmune disease, fused to intracellular signaling domains. Examples of the intracellular signaling domains of a CAAR include, without being limited to, T or NK receptor signaling domains such as CD137CD3ζ signaling domain.

In some embodiments, the CD8⁺CD45RC^{low/-} Treg cells of the invention are genetically modified and express at least one CAR, one CAAR and/or one native receptor linked to intracellular signaling molecules. Examples of CAR included, without being limited to, first generation CARs, second generation CARs, third generation CARs, CARs comprising more than three signaling domains (co-stimulatory domains and activation domain), and inhibitory CARs (iCARs).

According to the present invention, the extra-cellular domain of the CAR recognizing an antigen of interest may comprise a receptor, or a fragment of a receptor, which binds to said antigen, such as an antibody or an antigen-binding fragment thereof. According to the present invention, the extra-cellular domain of the CAR may comprise a human antibody or an antibody originating from any other species. As used herein, the term “antibody fragment” refers to at least one portion of an antibody that retains the ability to specifically interact with an epitope of an antigen. Examples of antibody fragments include, without being limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody.

In some embodiments, the CD8⁺CD45RC^{low/-} Treg cells of the invention are genetically modified and lack expression of a functional T cell receptor (TCR) and/or human leukocyte antigen (HLA), e.g., HLA class I and/or HLA class II. In some embodiments, the genetically modified CD8⁺CD45RC^{low/-} Treg cells of the invention lacking a functional TCR and/or HLA are allogeneic Tregs.

As used herein, the term “rapamycin compound” includes compounds having the rapamycin core structure as defined in U.S. Patent Application Publication No. 2003/0008923 (which is herein incorporated by reference), which may be chemically or biologically modified while still retaining mTOR inhibiting properties. In some embodiments, the rapamycin compound is rapamycin. The derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus. Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). Examples of 27-esters and ethers of rapamycin are disclosed in U.S. Pat. No. 5,256,790, which is hereby incorporated by reference in its entirety. Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference. Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein. Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD.

In some embodiments, the population of CD8⁺CD45RC^{low/-} Treg cells is cultured in presence of antigen-presenting cells (APCs). As used herein, the term “antigen-presenting cell(s)”, “APC” or “APCs” include both intact, whole cells as well as other molecules (all of allogeneic origin) which are capable of inducing the presentation of one or more antigens, preferably in association with class I MHC molecules, and all types of mononuclear cells which are capable of inducing an allogeneic immune response. Preferably whole viable cells are used as APCs. Examples of suitable APCs are discussed in detail below and include, but are not limited to, whole cells such as monocytes, macrophages, dendritic cells, monocyte-derived dendritic cells, macrophage-derived dendritic cells, B cells and myeloid leukaemia cells e.g. cell lines THP-1, U937, HL-60 or CEM-CM3.

Typically, the population of CD8⁺CD45RC^{low/-} Treg cells is cultured in an appropriate culture medium. As used herein, the term “medium” refers to a medium for maintaining a cell population, or culturing a cell population (e.g. “culture medium”) containing nutrients that maintain cell viability and support proliferation. The medium may contain any of the following in an appropriate combination: salt(s), buffer(s), amino acids, glucose or other sugar(s), antibiotics, serum or serum replacement, and other components such as growth factors, cytokines etc. Media ordinarily used for particular cell types are known to those skilled in the art. The medium of the invention may be based on a commercially available medium such as RPMI 1640 from Invitrogen.

In some embodiments, the rapamycin compound is added in culture medium at day 0 and 7 of the expansion or after 14 days. In some embodiments, the rapamycin compound is added in culture medium at day 0 and 7 of the expansion and another immune suppressive drug such as methylprednisolone is added to the culture medium.

In some embodiments, the culture medium comprises an amount of rapamycin of about 45 ng/ml. As used herein, the term ‘about’ as used herein when referring to a measurable value such as a parameter, an amount, a temporal duration, and the like, is meant to encompass variations of +/−20% or less, preferably +/−15% or less, more preferably +/−10% or less, and still more preferably +/−5% or less of and from the specified value, insofar such variations are appropriate to perform in the disclosed invention. It is to be understood that the value to which the modifier ‘about’ refers is itself also specifically, and preferably, disclosed. In some embodiments, the culture medium comprises an amount of rapamycin of 35, 36, 37, 38, 39, 40, 41, 42, 43, 44, 45, 46, 47; 48; 49, 50, 51, 52, 53, 54, or 55 ng/ml.

In some embodiments, cytokines, preferably IL-2 and/or IL-15, are added to the culture medium at day 0 of culture. In some embodiments, cytokines, preferably IL-2 and/or IL-15, are further added to the culture medium once, twice or three times or more, for example at day 1, 2, 3, 4, 5, 6, 7, 8, 9, 10, 11, 12, 13, 14, 15, 16, 17, 18, 19 and/or 20. In some embodiments, cytokines, preferably IL-2 and/or IL-15, are added to the culture medium at day 0 and at day 5, 6, 7, or 8 of culture. In some embodiments, cytokines, preferably IL-2 and/or IL-15, are added to the culture medium at day 0 and every 2, 3 or 4 days until the end of the culture.

In some embodiments of the method of the present invention, antibodies anti-CD3 and/or antibodies anti-CD28 are added to the culture medium at day 0, 1, 2, 3, 4, 5, 6, 7, 8, 9, 10, 11, 12, 13, 14, 15, 16, 17, 18, 19 and/or 20 culture, preferably at day 0 and/or at day 11, 12, 13, 14 and/or 15. In some embodiments, 0.1 to 10 μg/ml, preferably 0.25 to 4 μg/ml, more preferably 1 μg/ml of anti-CD3 antibody and/or 0.1 to 10 μg/ml, preferably 0.25 to 4 μg/ml, more preferably 1 μg/ml of anti-CD28 antibody are added to the culture medium.

Typically, the culture shall be carried out for at least 12 days, such as, for example, for between 12 days and not more than 6-8 weeks, preferably 14 days.

The method of the present invention is particular useful for adoptive T cell transfer for preventing or reducing transplant rejection or GVHD.

As used herein, the term “preventing or reducing transplant rejection” is meant to encompass prevention or inhibition of immune transplant rejection, as well as delaying the onset or the progression of immune transplant rejection. The term is also meant to encompass prolonging survival of a transplant in a patient, or reversing failure of a transplant in a patient. Further, the term is meant to encompass ameliorating a symptom of an immune transplant rejection, including, for example, ameliorating an immunological complication associated with immune rejection, such as for example, interstitial fibrosis, chronic graft arteriosclerosis, or vasculitis. As used herein, the term “transplant rejection” encompasses both acute and chronic transplant rejection. “Acute rejection” is the rejection by the immune system of a tissue transplant recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, cyclosporin and the like. “Chronic rejection” generally occurs in humans within several months to years after engraftment, even in the presence of successful immunosuppression of acute rejection. Fibrosis is a common factor in chronic rejection of all types of organ transplants. The term “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species, for example, a baboon heart being transplanted into a human host. In some embodiments the donor of the transplant is a human. The donor of the transplant can be a living donor or a deceased donor, namely a cadaveric donor. In some embodiments, the transplant is an organ, a tissue or cells. As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus. As used herein, the term “tissue” refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue. In a particular embodiment of the invention, the transplant is a cardiac allotransplant. As used herein, the term “cells” refers to a composition enriched for cells of interest, preferably a composition comprising at least 30%, preferably at least 50%, even more preferably at least 65% of said cells. In some embodiments the cells are selected from the group consisting of multipotent hematopoietic stem cells derived from bone marrow, peripheral blood, or umbilical cord blood; or pluripotent (i.e. embryonic stem cells (ES) or induced pluripotent stem cells (iPS)) or multipotent stem cell-derived differentiated cells of different cell lineages such as cardiomyocytes, beta-pancreatic cells, hepatocytes, neurons, etc. . . . .

In some embodiments, the cell composition is used for allogeneic hematopoietic stem cell transplantation (HSCT) and thus comprises multipotent hematopoietic stem cells, usually derived from bone marrow, peripheral blood, or umbilical cord blood. HSCT can be curative for patients with leukemia and lymphomas. However, an important limitation of allogeneic HCT is the development of graft versus host disease (GVHD), which occurs in a severe form in about 30-50% of humans who receive this therapy. The population of Tregs cells as prepared by the method of the present invention is thus particularly suitable for preventing or reducing Graft-versus-Host-Disease (GvHD). Accordingly, in some embodiments, the patient in need thereof is affected with a disease selected from the group consisting of acute myeloid leukemia (AML); acute lymphoid leukemia (ALL); chronic myeloid leukemia (CML); myelodysplasia syndrome (MDS)/myeloproliferative syndrome; lymphomas such as Hodgkin and non-Hodgkin lymphomas, chronic lymphatic leukemia (CLL) and multiple myeloma.

In some embodiments, the cell composition of the present invention is particularly suitable of the treatment of genetic diseases in which activation of the immune system is involved and wherein inhibition of immune responses would be beneficial. Examples of said diseases include but are not limited to monogenic genetic diseases affecting the immune system associated to autoimmunity, such as IPEX (immunodysregulation polyendocrinopathy enteropathy X-linked syndrome) and APECED (autoimmune polyendocrinopathy-candidiasis-ectodermal dystrophy), B cell primary immunodeficiencies, Muckle-Wells syndrome, mixed autoinflammatory and autoimmune syndrome, NLRP12-associated hereditary periodic fever syndrome, tumor necrosis factor receptor 1 associated periodic syndrome) and monogenic hereditary diseases, such as Duchenne muscular dystrophy (DMD), cystic fibrosis, lysosomal diseases and alpha1-anti-trypsin deficiency.

A further object of the present invention relates to a method of preventing or reducing transplant rejection or GVHD comprising administering a therapeutically effective combination of a population of CD8+CD45RC^{low/-} Tregs in combination with a rapamycin compound.

A further object of the present invention relates to a method of treating a genetic disease (as above described) in a patient in need thereof comprising administering a therapeutically effective combination of a population of CD8+CD45RC^{low/-} Tregs in combination with a rapamycin compound.

As used herein, the term “therapeutically effective combination” as used herein refers to an amount the population of Tregs together with the amount of the rapamycin compound that is sufficient to prevent or reduce transplant rejection or GVHD. The “therapeutically effective amount” is determined using procedures routinely employed by those of skill in the art such that an “improved therapeutic outcome” results. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the population of CD8+CD45RC^{low/-} Tregs and the rapamycin compound are administered to the subject in the form of a pharmaceutical composition. Typically, the Population of Tregs and the rapamycin compound may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The population of Tregs and the rapamycin compound can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The inventors also showed that a combination of cyclosporine and methylprednisolone increases the immunosuppressive capacity of the population of CD8⁺CD45RC^{low/-} Tregs.

Accordingly a further object of the present invention relates to a method of increasing the immunosuppressive capacity of a population of CD8⁺CD45RC^{low/-} Tregs comprising culturing the population of CD8⁺CD45RC^{low/-} Tregs in presence of a combination of cyclosporine and methylprednisolone. All the embodiments described for the culture of said population in presence of rapamycin apply mutatis mutandis to the combination of cyclosporine and methylprednisolone.

A further object of the present invention also relates to a method of preventing or reducing transplant rejection or GVHD comprising administering a therapeutically effective combination of a population of CD8+CD45RC^{low/-} Tregs, cyclosporine and methylprednisolone. All the embodiments for the method of preventing or reducing transplant rejection or GVHD described with rapamycin apply mutatis mutandis to the combination of cyclosporine and methylprednisolone.

As used herein, the term “cyclosporine” has its general meaning in the art and refers to cyclosporin A, derivatives of cyclosporin A, salts of cyclosporin A and the like and mixtures thereof.

As used herein, the term “methylprednisolone” has its general meaning in the art and refers to 6α, 11β)-11,17,21-trihydroxy-6-methyl-pregna-1,4-diene-3,20-dione.

The invention will be further illustrated by the following figures and examples. However, these examples and figures should not be interpreted in any way as limiting the scope of the present invention.

### FIGURES

FIG. 1. CD8⁺CD45RC^{low/-} Tregs expansion for cell therapy. CD8⁺CD45RC^{low/-} Tregs were sorted from fresh blood of HVs, expanded for 14 d with anti-CD3 and anti-CD28 MAbs, then cultured for 7 d in presence of allogeneic APCs in medium supplemented or not with IL-2 and IL-15 and immunosuppressive drugs and assessed for survival. Values >1 means proliferation of cells. n=7.

FIG. 2. Effect of supplementation of culture medium with rapamycin on expansion yield, phenotype and suppressive activity of Tregs A. Results are expressed as number of cells harvested at days 7, 14 and 20 normalized to cell number plated at day 0, after culture in medium supplemented with rapamycin or not. B-D. Tregs cultured for 7 (B), 14 (C) or 20 (D) days in presence or absence of rapamycin were analyzed for Tregs associated markers expression. E. Left. Results are expressed as % of suppression mediated by Tregs after culture in presence of rapamycin normalized to % of suppression mediated by Tregs after culture in absence of rapamycin. Right. Representative histogram of responder T cells proliferation in response to allogeneic APCs in absence (w/o Tregs) or presence of Tregs cultured in absence (named w NT expanded Tregs) or in presence of rapamycin (named w rapa-expanded Tregs).

FIG. 3: Effect of supplementation of culture medium with CsA, MPA, MPr or tacrolimus on expansion yield and suppressive activity of TregsA. Results are expressed as number of cells harvested at days 7 normalized to cell number plated at day 0, after culture in medium supplemented with one IS drug or not. B-D. Tregs were cultured for 7 days in presence or absence of one IS drug, then cultured with the same drug or a different one, and analyzed for expansion yield (B) and suppressive activity on CD4+CD25− effector T cells stimulated with allogeneic APCs (C-D). B. Results are expressed as number of cells harvested at days 14 normalized to cell number plated at day 0. C. Results are expressed as % of suppression mediated by Tregs after culture with IS drugs normalized to % of suppression mediated by Tregs after culture without any IS drug. D. Results are expressed as mean of suppressive score in function of mean of expansion score for each IS drugs combination. 1:1 means suppression and expansion scores obtained with Tregs expanded without any IS drug.

FIG. 4. Effect of Rapamycin, CsA, MPA, MPr or tacrolimus on allogeneic activation and phenotype of Tregs. A. Results are expressed as number of cells harvested at days 21 normalized to cell number plated at day 0, after culture in medium supplemented with Is drug or not. B. Tregs were analyzed for Tregs associated markers expression at day 21 (14 days drug free expansion and 7 days IS-supplemented medium culture).

### EXAMPLE 1

Material & Methods

CD8⁺CD45RC^{low/-} Tregs from fresh or thawed PBMCs were seeded at 3×10⁵/ml in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml), and were stimulated with coated anti-CD3 mAb (1 μg/ml), soluble anti-CD28 mAb (1 μg/ml) and/or allogeneic APCs at 1:4 Tregs: APCs ratio. At day 7, expanded cells were diluted at 1.5×10⁵/ml and stimulated again with coated anti-CD3 and soluble anti-CD28 mAbs (1 μg/ml each). IL-2 and IL-15 cytokines were freshly added at days 0, 7, 10 and 12 Immunosuppressive drugs, such as cyclosporine A (45 ng/ml), rapamycin (45 ng/ml), methylprednisolone (500 pg/ml), tacrolimus (2 ng/ml) or mycophenolate mofetil (1 μg/ml) were added in culture medium at day 0 and 7 of the expansion or after 14 days drug-free expansion to assess their toxicity and effect on suppression on CD8⁺CD45RC^{low/-} Tregs. At day 14, expanded Tregs were washed with PBS before use. Suppressive activity was tested on CD8⁺CD45RC^{low/-} Tregs expanded more than 10 fold in 7 days. For long-term expansion, CD8⁺CD45RC^{low/-} Tregs and CD4⁺CD25^{high}CD127^{low/-} Tregs were stimulated again with coated anti-CD3 (1 μg/ml) and soluble anti-CD28 MAbs (1 μg/ml) at days 14 and 21 and IL-2 and IL-15 cytokines were freshly added every 2 days from day 7 to 28. Culture medium 1× cytokines (IL-2 (1000 U/ml) and IL-15 (10 ng/ml)) was added when required.

Results

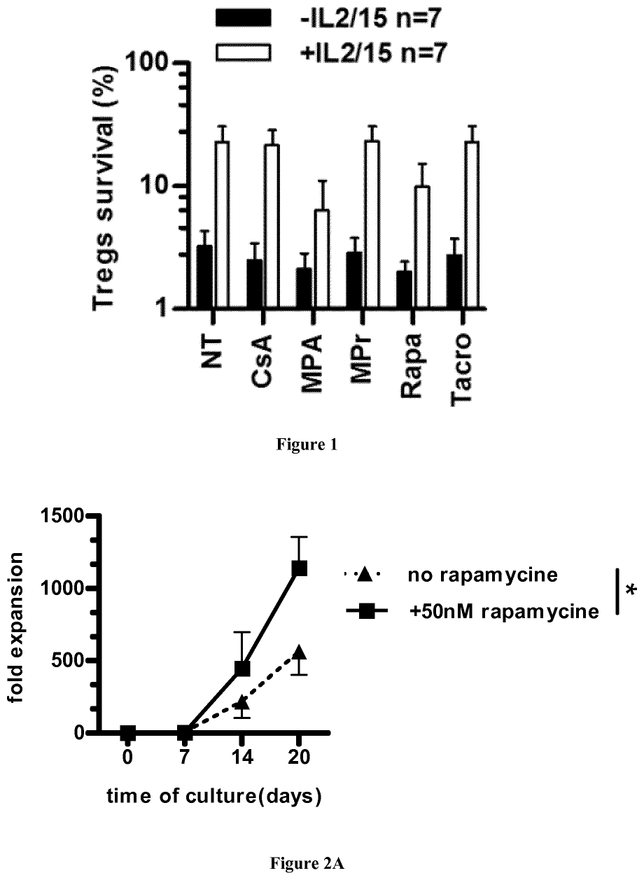

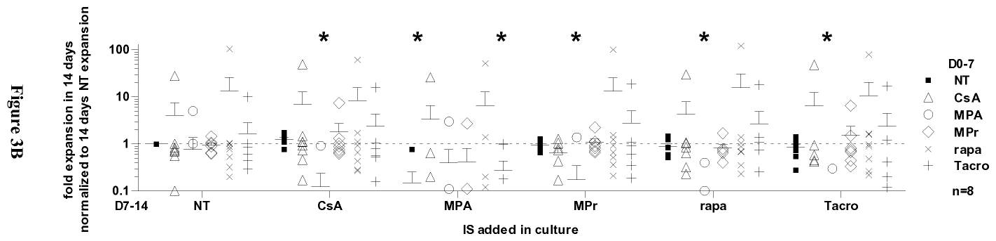

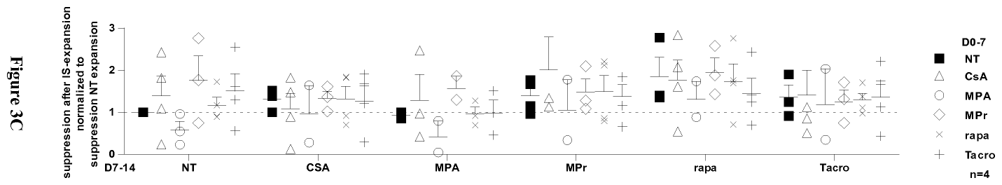

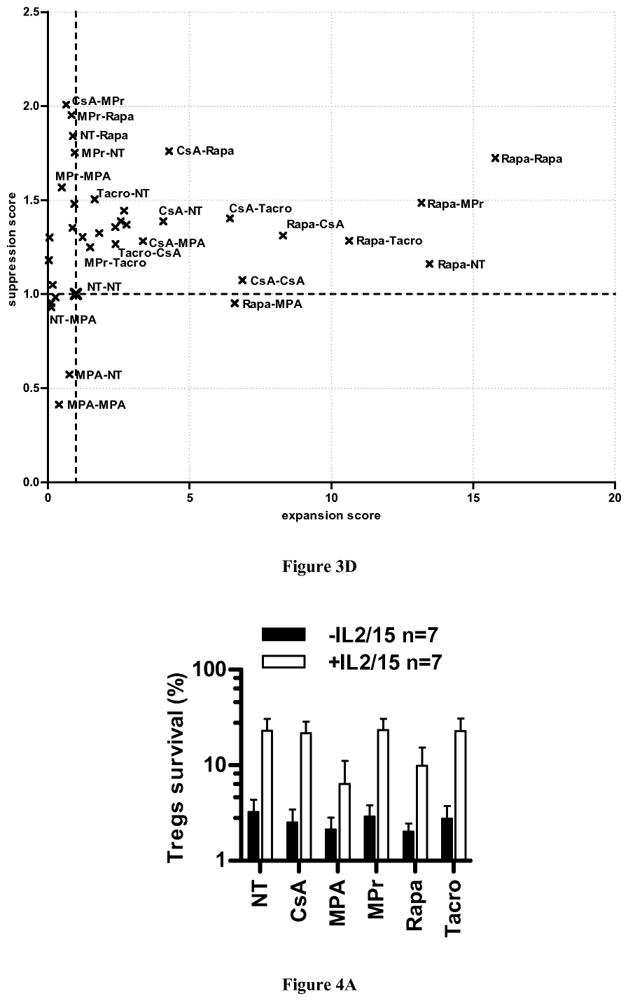

We tested the effect of immunosuppressive drugs (at concentrations used in the clinic) on CD8⁺CD45RC^{low/-} Tregs survival 7 days following the 14 days expansion (FIG. 1) or on the expansion yield and suppressive function score of CD8⁺CD45RC^{low/-} Tregs during the 14 days expansion (FIG. 3D). To test the effect of the immunosuppressors on the in vitro expansion, they were added either alone during 14 days or subsequently (i.e. day 0 to 7 cyclosporine A (CsA), then day 7 to 14 methylprednisolone (MPr) is labeled CsA-MPr in the upper left corner of FIG. 3D) or without any immunosuppressor (NT). We observed that 14 days expansion in presence of only rapamycin (Rapa) was the most efficient to increase both expansion fold and suppression capacity and first rapamycin and followed by methylprednisolone was also an efficient combination whereas the presence of mycophenolate mofetil (MPA) inhibited the expansion by rapamycin and decreased suppression (FIG. 3D), therefore, rapamycin might be beneficial to improve expansion and function of CD8⁺CD45RC^{low/-} Tregs in vitro but also in vivo.

### EXAMPLE 2

Methods: Fresh blood was taken from 1 healthy volunteer, PBMCs were isolated by Ficoll centrifugation, washed in PBS, adjusted at 2×10⁸ PBMC/ml in PBS-FCS-EDTA and were incubated with anti-CD3-PeCy7, anti CD4-PerCPCy5.5 and anti-CD45RC FITC 30′ 4° C. Cells were washed with PBS-FCS-EDTA, filtered on 60 μm tissue, labeled with Dapi and FACS Aria sorted on lymphocyte morphology, exclusion of doublet cells, and DAPI⁻CD3⁺CD4⁻CD45RC^{low/-} expression. After sorting, cells were washed in medium, plated at 10⁶Tregs/well/3 ml in p6 plate previously coated with anti-CD3 (OKT3 clone, 1 μg/ml in PBS, 1 ml/well, 1 h at 37° C. then washed with PBS 3 times), in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml) anti-CD28 mAbs (clone CD28.2, soluble, 1 μg/ml), and supplemented or not with 50 nM rapamycin. At days 7 and 14, Tregs were harvested, counted, washed, and plated at 5×10⁵Tregs/well/3 ml in p6 plate previously coated with anti-CD3 (OKT3 clone, 1 μg/ml in PBS, 1 ml/well, 1 h at 37° C. then washed with PBS 3 times), in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml), anti-CD28 mAbs (clone CD28.2, soluble, 1 μg/ml), and supplemented or not with 50 nM rapamycin. From day 7 to 20, cytokines were freshly added every 2 days and fresh medium (RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml)) was added when required, depending on proliferation rate. Expanded Tregs were harvested at day 14 for suppressive activity assessment. PBMCs from the same HV donor were thawed, washed in PBS, adjusted at 2×10⁸ PBMC/ml in PBS-FCS-EDTA and were incubated with anti-CD3-PeCy7, anti CD4-PerCPCy5.5, and anti-CD25-APC-Cy7 30′ 4° C. Cells were washed with PBS-FCS-EDTA, filtered on 60 μm tissue, labeled with Dapi and FACS Aria sorted on lymphocyte morphology, exclusion of doublet cells, and DAPI-CD3⁺CD4±CD25⁻ expression, and CFSE labeled. APCs were obtained by CD3⁺ cells depletion and 35Gy irradiation. Expanded Tregs were plated at 1:1:1 Tregs:Teff:APCs ratio in RPMI 1640 medium supplemented with 10% AB serum, Penicillin (100 U/ml), Streptomycin (0.1 mg/ml), Sodium pyruvate (1 mM), Glutamine (2 mM), Hepes Buffer (1 mM), and non-essential amino acids (1×). After 5 days culture, Teff proliferation was analyzed by CFSE analysis in DAPI⁻CD4±CD3⁺ T cells.

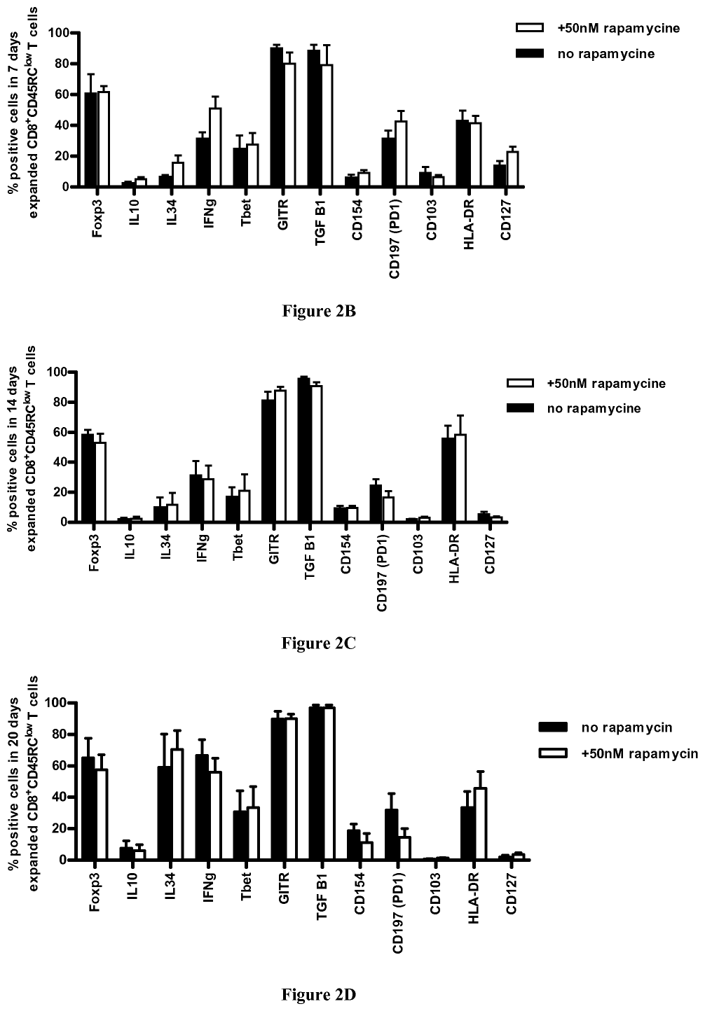

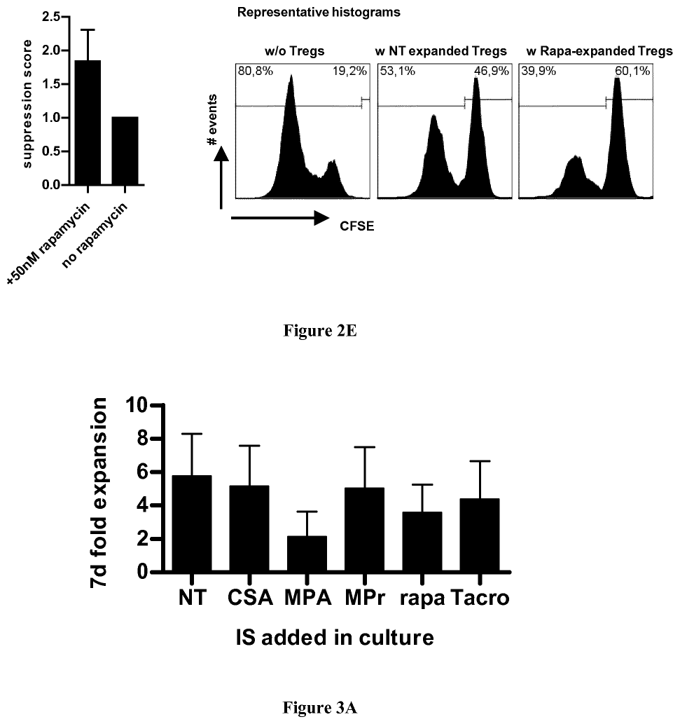

Results: Supplementation of culture medium for Tregs culture with rapamycin improved expansion yield and suppressive function without affecting cell phenotype (FIG. 2).

### EXAMPLE 3

Methods: Fresh blood was taken from 1 healthy volunteer, PBMCs were isolated by Ficoll centrifugation, washed in PBS, adjusted at 2×10⁸ PBMC/ml in PBS-FCS-EDTA and were incubated with anti-CD3-PeCy7, anti CD4-PerCPCy5.5 and anti-CD45RC FITC 30′ 4° C. Cells were washed with PBS-FCS-EDTA, filtered on 60 μm tissue, labeled with Dapi and FACS Aria sorted on lymphocyte morphology, exclusion of doublet cells, and DAPI-CD3⁺CD4⁻CD45RC^{low/-} expression. After sorting, cells were washed in medium, plated at 10⁶Tregs/well/3 ml in p6 plate previously coated with anti-CD3 (OKT3 clone, 1 μg/ml in PBS, 1 ml/well, 1 h at 37° C. then washed with PBS 3 times), in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml), anti-CD28 mAbs (clone CD28.2, soluble, 1 μg/ml), and supplemented or not with an immunosuppressive drug (cyclosporine A (45 ng/ml), rapamycin (45 ng/ml), methylprednisolone (500 pg/ml), tacrolimus (2 ng/ml) or mycophenolate mofetil (1 μg/ml)). At days 7, Tregs were harvested, counted, washed, and plated at 5×10⁵Tregs/well/3 ml in p6 plate previously coated with anti-CD3 (OKT3 clone, 1 μg/ml in PBS, 1 ml/well, 1 h at 37° C. then washed with PBS 3 times), in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml), anti-CD28 mAbs (clone CD28.2, soluble, 1 μg/ml), and supplemented or not with an immunosuppressive drug (cyclosporine A (45 ng/ml), rapamycin (45 ng/ml), methylprednisolone (500 pg/ml), tacrolimus (2 ng/ml) or mycophenolate mofetil (1 μg/ml)), the same drug as from day 0 to 7 or a different one. At days 10 and 12, cytokines were freshly added. Expanded Tregs were harvested at day 14 for suppressive activity assessment. PBMCs from the same HV donor were thawed, washed in PBS, adjusted at 2×10⁸ PBMC/ml in PBS-FCS-EDTA and were incubated with anti-CD3-PeCy7, anti CD4-PerCPCy5.5, and anti-CD25-APC-Cy7 30′ 4° C. Cells were washed with PBS-FCS-EDTA, filtered on 60 μm tissue, labeled with Dapi and FACS Aria sorted on lymphocyte morphology, exclusion of doublet cells, and DAPI-CD3⁺CD4±CD25⁻ expression, and CFSE labeled. APCs were obtained by CD3⁺ cells depletion and 35Gy irradiation. Expanded Tregs were plated at 1:1:1 Tregs:Teff:APCs ratio in RPMI 1640 medium supplemented with 10% AB serum, Penicillin (100 U/ml), Streptomycin (0.1 mg/ml), Sodium pyruvate (1 mM), Glutamine (2 mM), Hepes Buffer (1 mM), and non-essential amino acids (1×). After 5 days culture, Teff proliferation was analyzed by CFSE analysis in DAPI⁻CD4±CD3⁺ T cells.

Results: Supplementation of culture medium for Tregs culture with rapamycin, CsA, or Tacrolimus improved expansion yield and suppressive activity of Tregs, supplementation with MPA is deleterious for activity and expansion, and with methylprednisolone improved function (FIG. 3).

### EXAMPLE 4

Methods: Fresh blood was taken from 1 healthy volunteer, PBMCs were isolated by Ficoll centrifugation, washed in PBS, adjusted at 2×10⁸ PBMC/ml in PBS-FCS-EDTA and were incubated with anti-CD3-PeCy7, anti CD4-PerCPCy5.5 and anti-CD45RC FITC 30′ 4° C. Cells were washed with PBS-FCS-EDTA, filtered on 60 μm tissue, labeled with Dapi and FACS Aria sorted on lymphocyte morphology, exclusion of doublet cells, and DAPI-CD3⁺CD4⁻CD45RC^{low/-} expression. After sorting, cells were washed in medium, plated at 10⁶Tregs/well/3 ml in p6 plate previously coated with anti-CD3 (OKT3 clone, 1 μg/ml in PBS, 1 ml/well, 1 h at 37° C. then washed with PBS 3 times), in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml), anti-CD28 mAbs (clone CD28.2, soluble, 1 μg/ml). At days 7, Tregs were harvested, counted, washed, and plated at 5×10⁵Tregs/well/3 ml in p6 plate previously coated with anti-CD3 (OKT3 clone, 1 μg/ml in PBS, 1 ml/well, 1 h at 37° C. then washed with PBS 3 times), in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), IL-2 (1000 U/ml) and IL-15 (10 ng/ml), anti-CD28 mAbs (clone CD28.2, soluble, 1 μg/ml). At days 10 and 12, cytokines were freshly added. At day 14, expanded Tregs were harvested and plated at 5×10⁵Tregs/well/200 μl with allogeneic APCs (Tregs:APC 1:4) in p96 plate in RPMI1640 medium supplemented with 10% AB serum, Penicillin (100 U/ml), Streptomycin (0.1 mg/ml), Sodium pyruvate (1 mM), Glutamine (2 mM), Hepes Buffer (1 mM), non-essential amino acids (1×), and supplemented or not with an immunosuppressive drug (cyclosporine A (45 ng/ml), rapamycin (45 ng/ml), methylprednisolone (500 pg/ml), tacrolimus (2 ng/ml) or mycophenolate mofetil (1 μg/ml)) and with or without cytokines (IL-2 (1000 U/ml) and IL-15 (10 ng/ml) added at days 14, 16, 18 and 20). Tregs were analyzed at day 21 for survival/expansion yield and phenotype.

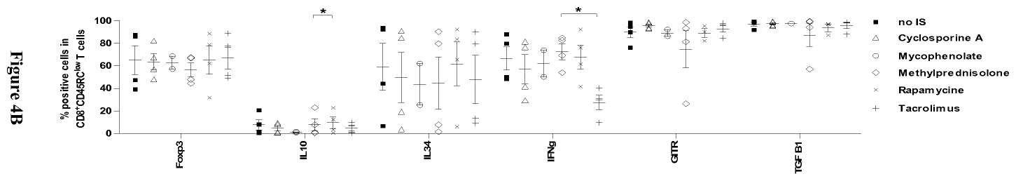

Results: Supplementation of culture medium for Tregs culture did not affect Tregs survival, neither Tregs proliferation in response to allogeneic APCs (FIG. 5). Tacrolimus and methylprednisolone significantly decreased IFNg and CD154 expression by Tregs respectively compared to drug free medium (FIG. 4).

### REFERENCES

Throughout this application, various references describe the state of the art to which this invention pertains. The disclosures of these references are hereby incorporated by reference into the present disclosure.
1. Nankivell, B. J., et al. The natural history of chronic allograft nephropathy. N Engl J Med 349, 2326-2333 (2003).
2. Moreau, A., Alliot-Licht, B., Cuturi, M. C. & Blancho, G. Tolerogenic dendritic cell therapy in organ transplantation. Transpl Int (2016).
3. Clement, M., et al. Control of the T follicular helper-germinal center B-cell axis by CD8(+) regulatory T cells limits atherosclerosis and tertiary lymphoid organ development. Circulation 131, 560-570 (2015).
4. Singer, B. D., King, L. S. & D'Alessio, F. R. Regulatory T cells as immunotherapy. Frontiers in Immunology 5, 46 (2014).
5. Tsang, J. Y., et al. The potency of allospecific Tregs cells appears to correlate with T cell receptor functional avidity. Am J Transplant 11, 1610-1620 (2011).
6. Tsang, J. Y., et al. Conferring indirect allospecificity on CD4+CD25+ Tregs by TCR gene transfer favors transplantation tolerance in mice. J Clin Invest 118, 3619-3628 (2008).
7. Sagoo, P., et al. Human regulatory T cells with alloantigen specificity are more potent inhibitors of alloimmune skin graft damage than polyclonal regulatory T cells. Sci Transl Med 3, 83ra42 (2011).
8. Picarda, E., Anegon, I. & Guillonneau, C. T-cell receptor specificity of CD8(+) Tregs in allotransplantation. Immunotherapy 3, 35-37 (2011).
9. Guillonneau, C., et al. CD40Ig treatment results in allograft acceptance mediated by CD8CD45RC T cells, IFN-gamma, and indoleamine 2,3-dioxygenase. J Clin Invest 117, 1096-1106 (2007).
10. Vuddamalay, Y. & van Meerwijk, J. P. CD28- and CD28lowCD8+ Regulatory T Cells: Of Mice and Men. Frontiers in Immunology 8, 31 (2017).
11. Wang, Y. M. & Alexander, S. I. CD8 regulatory T cells: what's old is now new. Immunology and Cell Biology 87, 192-193 (2009).
12. Hori, S., Nomura, T. & Sakaguchi, S. Control of regulatory T cell development by the transcription factor Foxp3. Science 299, 1057-1061 (2003).
13. Fontenot, J. D., Gavin, M. A. & Rudensky, A. Y. Foxp3 programs the development and function of CD4+CD25+ regulatory T cells. Nat Immunol 4, 330-336 (2003).
14. Guillonneau, C., Picarda, E. & Anegon, I. CD8+ regulatory T cells in solid organ transplantation. Curr Opin Organ Transplant 15, 751-756 (2010).
15. Niederkorn, J. Y. Emerging concepts in CD8(+) T regulatory cells. Curr Opin Immunol 20, 327-331 (2008).
16. Liu, J., Chen, D., Nie, G. D. & Dai, Z. CD8(+)CD122(+) T-Cells: A Newly Emerging Regulator with Central Memory Cell Phenotypes. Frontiers in Immunology 6, 494 (2015).

1-15. (canceled)

16. A method of increasing expansion and immunosuppressive capacity of a population of CD8+CD45RC^{low/-} Treg cells, wherein said method comprises culturing the population of CD8+CD45RC^{low/-} Treg cells in the presence of at least one immunosuppressive drug selected from the group consisting of a rapamycin compound and a combination of cyclosporine and methylprednisolone.

17. The method of claim 16, wherein the population of CD8+CD45RC^{low/-} Treg cells is genetically modified to encode a T cell receptor or a subunit or functional equivalent thereof.

18. The method of claim 16, wherein the population of CD8+CD45RC^{low/-} Treg cells is genetically modified to encode a chimeric antigen receptor (CAR) specific to an antigen of interest or a chimeric autoantibody receptor (CAAR) comprising an auto-antigen.

19. The method of claim 16, wherein the population of CD8+CD45RC^{low/-} Treg cells is genetically modified and lacks expression of a functional T cell receptor (TCR) and/or human leukocyte antigen (HLA).

20. The method of claim 16, wherein the population of CD8+CD45RC^{low/-} Treg cells is genetically modified and lacks expression of HLA class I and/or HLA class II.

21. The method of claim 16, wherein the population of CD8+CD45RC^{low/-} Treg cells is allogeneic Tregs vis-à-vis the recipient patient to be treated.

22. The method of claim 16, wherein the population of CD8+CD45RC^{low/-} Treg cells is cultured in the presence of antigen-presenting cells.

23. The method of claim 16, wherein the rapamycin compound is added in the culture medium at day 0 and 7 of culture or is added in the culture medium at day 14 of culture.

24. The method of claim 16, wherein the rapamycin compound is added in the culture medium at day 0 and 7 of culture and another immune suppressive drug is added to the culture medium.

25. The method of claim 24, wherein the other immune suppressive drug is methylprednisolone.

26. The method of claim 16, wherein the culture medium comprises an amount of rapamycin of about 45 ng/ml.

27. The method of claim 16, wherein cytokines are further added to the culture medium once, twice or three times or more.

28. The method of claim 27, wherein said cytokines are IL-2 and/or IL-15.

29. The method of claim 27, wherein said cytokines are added to the culture medium at day 1, 2, 3, 4, 5, 6, 7, 8, 9, 10, 11, 12, 13, 14, 15, 16, 17, 18, 19 and/or 20 of culture.

30. The method of claim 16, wherein an anti-CD3 antibody and/or an anti-CD28 antibody is added to the culture medium at day 0, 1, 2, 3, 4, 5, 6, 7, 8, 9, 10, 11, 12, 13, 14, 15, 16, 17, 18, 19 and/or 20 of culture.

31. The method of claim 30, wherein said anti-CD3 antibody and/or said anti-CD28 antibody is added to the culture medium at day 0 and/or at day 11, 12, 13, 14 and/or 15 of culture.

32. The method of claim 16, wherein the culture is carried out for at least 12 days.

33. A method of preventing or reducing transplant rejection or GVHD in a patient in need thereof comprising administering to the patient a therapeutically effective amount of a population of CD8+CD45RC^{low/-} Treg cells in combination with at least one immunosuppressive drug selected from the group consisting of a rapamycin compound and a combination of cyclosporine and methylprednisolone.

34. A method of treating a genetic disease in a patient in need thereof comprising administering to the patient a therapeutically effective amount of a population of CD8+CD45RC^{low/-} Treg cells in combination with a rapamycin compound wherein the genetic disease is selected from the group consisting of a monogenic genetic disease affecting the immune system associated to autoimmunity, and a monogenic hereditary disease.

35. The method of claim 34, wherein the monogenic genetic disease affecting the immune system associated to autoimmunity is selected from the group consisting of IPEX (immunodysregulation polyendocrinopathy enteropathy X-linked syndrome), APECED (autoimmune polyendocrinopathy-candidiasis-ectodermal dystrophy), B cell primary immunodeficiencies, Muckle-Wells syndrome, mixed autoinflammatory and autoimmune syndrome, NLRP12-associated hereditary periodic fever syndrome and tumor necrosis factor receptor 1 associated periodic syndrome, and wherein the monogenic hereditary disease is selected from the group consisting of Duchenne muscular dystrophy (DMD), cystic fibrosis, lysosomal diseases and alpha1-anti-trypsin deficiency.
